(19) United States
(12) Patent Application Publication
(10) Pub. No.: US 2023/0145444 A1
(43) Pub. Date: May 11, 2023

(54) IMAGING USING CAVITATION BUBBLES

(71) Applicant: Siemens Healthcare GmbH, Erlangen (DE)

(72) Inventors: Alois Regensburger, Poxdorf (DE); Tobias Lenich, Nürnberg (DE)

(21) Appl. No.: 17/983,390

(22) Filed: Nov. 8, 2022

(30) Foreign Application Priority Data
Nov. 9, 2021 (DE) 10 2021 212 610.3

(51) Int. Cl. A61B 8/08; A61B 5/055

(52) U.S. Cl. CPC A61B 8/481; A61B 5/055

(57) ABSTRACT

An imaging method for mapping a human or animal tissue area includes generating a plurality of cavitation bubbles in a liquid in the tissue area by ultrasound pulses being irradiated into the tissue area by at least one ultrasound source. A center of a focus area of the irradiated ultrasound pulses is positioned within a first subarea of the tissue area. The first subarea is separated from a second subarea of the tissue area by a tissue boundary, and/or a flow of liquid is present in the first subarea. A spatial distribution and/or movement of the plurality of cavitation bubbles developing in the tissue area on account of a pressure field caused by the irradiated ultrasound pulses is mapped by an imaging modality.

This application claims the benefit of German Patent Application No. DE 10 2021 212 610.3, filed on Nov. 9, 2021, which is hereby incorporated by reference in its entirety.

### BACKGROUND

The present embodiments relate to an imaging method for mapping a human or animal tissue area and to a corresponding imaging device.

With medical imaging (e.g., when x-ray-based imaging methods, ultrasound-based imaging methods, or magnetic resonance tomography methods are carried out), contrast agent may be administered to a patient in order to improve the visibility of anatomical structures and/or dynamic processes. The administration of contrast agent may be unpleasant for patients or, in individual cases, cause unwanted side effects.

Histotripsy is a method that may be used inter alia for non-invasive tumor ablation, as described, for example, in the publication Q. Shibin et al., “Non-thermal histotripsy tumor ablation promotes abscopal immune responses that enhance cancer immunotherapy,” Journal for Immunotherapy of Cancer, 8 (1), 2020.

Extreme pressure differences that result in destruction of the cell structure are generated as a result of highly intensive ultrasound pulses that converge into a focus area. This is based on the formation and destruction of cavitation bubbles in liquids in the tissue, which are generated by differences in pressure. In the case of histotripsy, the configuration parameters for generating the ultrasound pulses, such as, for example, the pulse duration, pulse amplitude, or pause times between individual ultrasound pulses or between sequences of ultrasound pulses, are selected such that a high energy input that results in a significant expansion and ultimate implosion of the cavitation bubbles, without, however, causing a relevant heat development, takes place. The explosion of the cavitation bubbles is associated with shockwaves and accordingly high forces on surrounding tissue, which results in destruction of the cell structure.

Depending on the choice of configuration parameters, unlike with histotripsy, it is, however, also possible to generate cavitation bubbles and possibly to keep the cavitation bubbles stable without destroying the cavitation bubbles as a result of a further heat input. In this context, stable cavitation is sometimes contrasted with unstable cavitation, as is utilized in histotripsy.

In the publication S.-W. Ohl et al, “Bubbles with shockwaves and ultrasound: A review,” Interface Focus 5: 20150019, the authors describe the interaction of stable cavitation bubbles inter alia with ultrasound. It is described, for example, that on account of a low pressure forming in the focus area of an ultrasound converter, cavitation bubbles generated in a liquid move in the direction of the focus area or toward the center of the focus area.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a representation of anatomical structures and/or of liquid flows in a tissue during imaging procedures without using contrast agent for this purpose is improved.

The present embodiments are based on the idea of generating stable cavitation bubbles in the tissue and mapping spatial distribution and/or movement of the cavitation bubbles that results from the ultrasound-induced pressure field in the tissue, using an imaging modality.

According to one aspect of the present embodiments, an imaging method for mapping a human or animal tissue area is specified. A plurality of cavitation bubbles (e.g., stable cavitation bubbles) is generated in a liquid in the tissue area by ultrasound pulses being irradiated into the tissue area by at least one ultrasound source. A center of a focus area of the irradiated ultrasound pulses is positioned within a first subarea of the tissue area, where the first subarea is separated from a second subarea of the tissue area by a tissue boundary, and/or a flow of liquid is present in the first subarea. A spatial distribution and/or movement of the plurality of cavitation bubbles developing in the tissue area on account of a pressure field caused by the irradiated ultrasound pulses is mapped by an imaging modality.

The distribution and/or the movement of the plurality of cavitation bubbles may be caused, for example, by the pressure field alone or, if there is a flow of liquid, by the pressure field and the flow of liquid.

In order to generate the ultrasound pulses, the at least one ultrasound source may be controlled by a controller. The controller may, for example, adjust at least one configuration parameter for controlling the at least one ultrasound source such that cavitation bubbles are generated by the irradiated ultrasound pulses in the liquid, but are not destroyed by further energy input on account of the ultrasound pulses or further ultrasound pulses. The cavitation bubbles are therefore generated as stable cavitation bubbles.

The at least one configuration parameter may include a pulse duration, a pulse amplitude, a pause time between consecutive ultrasound pulses or between associated sequences of consecutive ultrasound pulses, a pulse duty factor or duty cycle of the ultrasound pulses, and so forth, for example.

By adjusting the at least one configuration parameter, it is possible to adjust the strength of the pressure differences generated in the tissue by the ultrasound pulse (e.g., in the focus area). The at least one configuration parameter may therefore be determined, for example, so that the at least one configuration parameter quantifies an effective energy or effective power introduced by the ultrasound pulses. In this way, a rate of generating the cavitation bubbles may be adapted, for example, by adjusting the at least one configuration parameter and the size of the generated cavitation bubbles may be restricted so that the generated cavitation bubbles remain substantially stable and are not intentionally made to collapse, as would be the case with histotripsy. The generation of the cavitation bubbles within the scope of an imaging method in accordance with the present embodiments therefore does not result in the destruction or permanent modification of tissue.

Tissue here and below may include both solid and also liquid tissue, such as, for example, blood or lymph or cellular fluid. The liquid in which the cavitation bubbles are generated is a liquid that is present in the tissue area.

The mapping of the spatial distribution and/or movement of the cavitation bubbles by the imaging modality includes, for example, the generation of one or more images, possibly in a time-resolved manner, which represent at least one part of the cavitation bubbles.

In the ideal case, the focus area of the ultrasound pulses may be punctiform. In reality, this always has a finite extent, however, so that the focus area may be described at least approximately by a three-dimensional geometric figure. The center of the focus area may lie, for example, in the center of the volume of the geometric figure. For example, the focus area may be described approximately by a sphere, an ellipsoid, or an ovoid.

The pressure field resulting from the irradiated ultrasound pulses is, for example, inhomogenous. As a rule, an extreme point of the pressure field is generated in the center or close to the center of the focus area. A pressure gradient, which results in the movement of the cavitation bubbles, is therefore generally present in the tissue area. Depending on the other conditions in the tissue area (e.g., whether other forces or flows are acting on the cavitation bubbles), a static spatial or approximately static spatial distribution of the cavitation bubbles may also develop. For example, the plurality of cavitation bubbles may move in the direction of the center of the focus area unless the cavitation bubbles are prevented from moving as a result of corresponding forces or structures in the tissue area (e.g., as a result of the tissue boundary). In other situations, a more or less permanent movement of the cavitation bubbles that may be mapped by the imaging modality (e.g., in a time-resolved manner) may develop.

In other words, the distribution and/or movement, which would otherwise develop on account of the pressure field, is influenced by the tissue boundary and/or the flow of the liquid (e.g., by additional forces acting on the cavitation bubbles as a result of the tissue boundary and/or the flow of liquid). These forces may be aligned arbitrarily with respect to the pressure field. A characteristic influencing of the distribution and/or movement of the cavitation bubbles therefore occurs, which is mapped by the imaging modality.

Accordingly, a characteristic arrangement or characteristic spatial distribution and/or characteristic movement of the cavitation bubbles within the tissue area, which may be mapped by the imaging modality, possibly in a time-resolved manner, is produced from the interaction between the anatomical structure and the flow ratios in the tissue area with the cavitation bubbles. In this way, the cavitation bubbles may be effectively used like a contrast agent, without a contrast agent actually having to be used, since it is possible to infer anatomical structures or the course of anatomical structures or flow properties of the liquid from the movements and/or the spatial distribution of the cavitation bubbles if the at least one configuration parameter is known.

The cavitation bubbles may be generated, for example, non-invasively by the ultrasound pulses and on account of the normalizing pressure ratios dissipate again without the application of ultrasound. Further, the cavitation bubbles may be generated selectively at points in the body that would not be reachable with conventional contrast agents administered through the blood.

In corresponding embodiments, the cavitation bubbles may be generated at least partly in the second subarea. Since the center of the focus area is located in the first subarea, the cavitation bubbles generally move in the direction of the tissue boundary. The tissue boundary may be such that the cavitation bubbles cannot pass through the tissue boundary. It may be possible to adapt a size of the cavitation bubbles accordingly in order to achieve this using the corresponding adaptation of the at least one configuration parameter.

In such a situation, the cavitation bubbles therefore adsorb to the tissue boundary and allow the tissue boundary to be localized by the mapping using the imaging modality. For example, the adsorbing cavitation bubbles have a different contrast to the surrounding tissue. By identifying the cavitation bubbles in the mapping (e.g., by a corresponding segmentation), the tissue boundary may be identified on one side of the distribution of the cavitation bubbles.

However, the cavitation bubbles may also be generated at least in part in the first subarea. If the liquid in the first subarea is subjected to a flow (e.g., if the first subarea is an interior of a blood vessel or suchlike), the cavitation bubbles in the first subarea are therefore also subjected to forces on account of the liquid flow and move accordingly. By analyzing the mapping (e.g., the time-resolved mapping) using the imaging modality, if the at least one configuration parameter is known, inferences may therefore be drawn as to the flow properties and/or material properties of the liquid in the first subarea (e.g., relating to a flow direction and/or a flow speed and/or a viscosity of the liquid and so forth).

It may also be that the tissue boundary contains openings or is passed through by vessels, for example, which lead from the first subarea through the tissue boundary into the second subarea or vice versa. If the cavitation bubbles are generated at least in part in the area of the opening or in the area of the vessel leading through the tissue boundary, it is therefore possible to also infer a movement direction of the liquid in the vessel and possibly also a flow speed from the result of the imaging. A distinction may therefore be made between vessels that transport liquid from the second area into the first subarea and vessels that transport liquid from the first subarea into the second subarea.

For example, the size of the generated cavitation bubbles (e.g., a diameter of the cavitation bubbles) may also be adapted by adapting the configuration parameters. In this way, it is possible to distinguish between small and large openings in the tissue boundary or between small and large vessels.

The tissue boundary may also be porous or permeable. The cavitation bubbles may then pass through or diffuse through the tissue boundary.

According to at least one embodiment of the imaging method, the mapping of the spatial distribution and/or movement of the plurality of cavitation bubbles includes an x-ray-based imaging (e.g., an x-ray tomography or computed tomography), a magnetic resonance tomography, or an ultrasound-based imaging.

In other words, the imaging modality is configured to be an x-ray based imaging modality (e.g., an x-ray tomography device or a computed tomography device), a magnetic resonance tomography device, or an ultrasound-based imaging modality.

The at least one ultrasound source for generating the ultrasound pulses in the tissue area, which generate the plurality of cavitation bubbles, is to be considered independently of the imaging modality, particularly if the imaging modality is an ultrasound-based imaging modality. The ultrasound-based imaging modality may include, for example, one or more further ultrasound sources or ultrasound converters that are designed or configured for imaging. The at least one ultrasound source for generating the cavitation bubbles is not necessarily designed to be used for imaging in the narrower sense (e.g., for mapping the tissue area).

For example, a histotripsy device that contains the at least one ultrasound source may be used. In other words, the at least one ultrasound source may be configured as at least one histotripsy converter.

This may be that the at least one ultrasound source may, in principle, also be used to carry out a histotripsy therapy. However, a corresponding adaptation of the at least one configuration parameter prevents the cavitation bubbles from being destroyed and accordingly the tissue from being intentionally destroyed. In other words, an imaging method of the present embodiments includes no histotripsy, even if the at least one ultrasound source may in principle be suitable for this. For example, in the context of an imaging method, the destruction of tissue should be avoided in principle.

According to at least one embodiment, the first subarea corresponds to an inner area of an object within the tissue area, and the second subarea corresponds to an outer area, within the tissue area, that surrounds the object at least in part. The object may be, for example, a tumor, a cyst, any other mass, a vessel (e.g., a blood vessel, or a vascular malformation (e.g., a nidus). In other words, the focus area is therefore centered in the inner area of the object, or the center of the focus area is positioned in the inner area of the object. As mentioned above, due to the resulting low pressure, this results in a force acting on the cavitation bubbles that points in the direction of the center of the focus area.

For example, the ultrasound pulses are generated such that the pressure field has a minimum in the inner area.

This is achieved, for example, by the at least one configuration parameter being adjusted accordingly and the center of the focus area being positioned accordingly as described.

According to at least one embodiment, the ultrasound pulses are generated such that at least one part of the plurality of cavitation bubbles is generated in the outer area.

In addition to the effectively input intensity or power as a result of the ultrasound pulses, whether cavitation bubbles are generated and whether these are stable or unstable also depends on the material properties in the tissue area (e.g., the liquid in which the cavitation bubbles are generated). Since the material parameters of the tissue in the inner area and in the outer area may differ from one another, it is therefore possible using a corresponding adaptation of the at least one configuration parameter and possibly the position of the center of the focus area to adjust the spatial area in which the cavitation bubbles are generated and/or the rate at which the cavitation bubbles are generated in a location-dependent manner.

Although the center of the focus area is located in the inner area of the object, the pressure field is nonetheless also generally not constant in the outer area. This results in a corresponding force in the direction of the center of the focus area also acting on cavitation bubbles in the outer area. Such cavitation bubbles may adsorb accordingly to the tissue boundary so that the tissue boundary may be shown in the imaging with improved visibility (e.g., by a flattening on one side of the cavitation bubbles).

The position of the center of the focus area and/or of the at least one configuration parameter may be adjusted, for example, such that cavitation bubbles are only generated in the outer area, but not, however, in the inner area or possibly in the inner area but only at a very much lower rate. This improves the contrast effect of the cavitation bubbles adsorbed to the outside of the tissue boundary in the imaging. However, even when cavitation bubbles are generated within the inner area (e.g., at a lower rate), the visibility of the tissue boundary in the imaging may nevertheless be improved since a force acts on the cavitation bubbles in the inner area. The force is generally directed away from the tissue boundary so that in the inner area in any case a depletion of cavitation bubbles at the tissue boundary is achieved.

According to at least one embodiment, based on a result of the mapping of the movement of the plurality of cavitation bubbles, a movement direction and/or a movement speed of one or more cavitation bubbles of the part of the plurality of cavitation bubbles generated in the outer area through a vessel is determined. The vessel extends through the tissue boundary and fluidically connects the inner area with the outer area.

The result of the mapping corresponds, for example, to a plurality of images (e.g., a sequence of images or an image sequence or video sequence) that map or represent the cavitation bubbles.

The movement direction or movement speed of the cavitation bubbles in the vessel directly reflects the flow direction or accordingly the flow speed and/or a viscosity of the liquid inside the vessel. It is therefore possible to infer the flow speed and/or flow direction and/or viscosity of the liquid from the movement speed and/or movement direction of the cavitation bubbles.

According to at least one embodiment, the ultrasound pulses are generated so that at least one part of the plurality of cavitation bubbles is generated in the inner area.

Similar to the description above, this is achieved by the positioning of the center of the focus area and the selection of the at least one configuration parameter.

Such embodiments are suitable, for example, if the object is a vessel (e.g., a blood vessel and/or a hollow organ). The cavitation bubbles in the vessel are namely subjected not only to the effect of the force that is generated by the pressure field resulting from the irradiated ultrasound pulses, but also to forces due to the liquid flowing in the vessel. The former force points in the direction of the center of the focus area, while the latter force points upstream with respect to the center likewise in the direction of the center, downstream of the center but away from the center. The forces therefore strengthen downstream of the center and attenuate downstream of the center.

This results in a characteristic asymmetric distribution of the cavitation bubbles in the inner area of the object. For example, the cavitation bubbles are carried further downstream away from the center of the focus area than upstream. If the cavitation bubbles move too far away from the center of the focus area, the pressure ratios normalize there, which results in the cavitation bubbles disappearing. By correspondingly selecting the at least one configuration parameter, an equilibrium condition may therefore develop by the forces on the cavitation bubbles being balanced in an area considered.

With a given flow speed and viscosity of the liquid, given configuration parameters for generating the ultrasound pulses and with a constant position of the center of the focus area, a characteristic stationary distribution of the cavitation bubbles may develop. This distribution is mapped by the imaging and may be analyzed accordingly in order to infer the flow properties (e.g., the flow speed and/or the viscosity using the configuration parameters). This may also be considered indirectly as a measure of a spatial extent of the vessel (e.g., the cross-section of the vessel).

In other words, in various embodiments, it is possible to determine the flow direction and/or flow speed and/or viscosity of the liquid in the inner area based on a result of the mapping of the spatial arrangement and/or the movement of the plurality of cavitation bubbles.

To this end, geometric properties of the cavitation bubbles or the distribution of the cavitation bubbles may be analyzed, for example. If there were no fluid flow, the expectation would be that the cavitation bubbles are distributed approximately symmetrically about the center of the focus area. The effect of the flow on the cavitation bubbles brings about an asymmetry with a flattening or compression of the distribution upstream of the center of the focus area and an extension or elongation of the distribution downstream of the center of the focus area. A teardrop distribution, for example, is therefore produced.

In various embodiments, the ultrasound pulses are then generated so that no cavitation bubbles are generated except in the outer area or cavitation bubbles are only generated at a lower rate than in the inner area.

In other embodiments, a part of the cavitation bubbles is generated in the outer area, and another part of the cavitation bubbles is generated in the inner area. In such embodiments, it is possible to observe both the tissue boundary and also the flow properties at the same time.

According to at least one embodiment, the first subarea is separated from the second subarea by the tissue boundary, and in the first subarea, there is a flow of liquid. The ultrasound pulses are generated so that at least one part of the plurality of cavitation bubbles is generated in the inner area.

For example, the position of the center of the focus area within the inner area may be moved and the flow direction and/or flow speed and/or viscosity within the inner area may be determined in a location-dependent manner (e.g., based on corresponding results of the mapping of the spatial arrangement and/or movement of the plurality of cavitation bubbles).

The flow direction, flow speed, or viscosity of the liquid may be location-dependent inside the vessel (e.g., if the cross-section of the vessel changes). This also acts on the spatial distribution and/or arrangement of the cavitation bubbles, when the center of the focus area is moved accordingly (e.g., along the vascular path, and thus along a longitudinal direction of the vessel). A spatially resolved determination of the flow speed and/or flow direction and/or viscosity of the liquid may therefore be achieved by changing the mapping of the spatial distribution and/or movement of the cavitation bubbles. The change may correspond to a change in at least one geometric parameter of the distribution of cavitation bubbles (e.g., with respect to a reference position). The at least one geometric parameter may relate, for example, to a density, an extent, a shape, an asymmetry, and so forth.

The focus area or a center of the focus may be moved in this case, for example, as a result of a movement of the at least one ultrasound source, a movement of the patient, or as a result of an electronic change in the focus position (e.g., without an actual movement of the at least one ultrasound source relative to the patient).

According to a further aspect of the present embodiments, an imaging device for mapping a human or animal tissue area is specified. The imaging device is configured to carry out an imaging method according to the present embodiments or carries out such an imaging method.

According to at least one embodiment of the imaging device, the imaging device includes an imaging modality and a controller. Further, the imaging device includes at least one ultrasound source, and the controller is configured to control the at least one ultrasound source in order to irradiate ultrasound pulses into the tissue area. The controller is configured to adjust at least one configuration parameter for controlling the at least one ultrasound source such that cavitation bubbles (e.g., stable cavitation bubbles) are generated by the irradiated ultrasound pulses in a liquid in the tissue area. The imaging device also has a positioning system for the at least one ultrasound source, which is configured to position a center of a focus area of the irradiated ultrasound pulses within a first subarea of the tissue area. The first subarea is separated from a second subarea of the tissue area by a tissue boundary, and/or a flow of liquid is present in the first subarea. The imaging modality is configured to map a spatial distribution developing in the tissue area on account of a pressure field caused by the irradiated ultrasound pulses and/or movement of the cavitation bubbles.

In various embodiments, the controller may also be configured to control the positioning system and/or the imaging modality. In alternative embodiments, the imaging device has one or more further controllers for controlling the imaging device and/or the imaging modality.

According to a further aspect of the present embodiments, a computer program with commands is specified. When the commands are executed by an imaging device of the present embodiments (e.g., by the controller of the imaging device and/or possibly the one or more further controllers of the imaging device), the commands cause the imaging device to carry out an imaging method of the present embodiments.

According to a further aspect of the present embodiments, a computer-readable storage medium (e.g., a non-transitory computer-readable storage medium) that stores a computer program according to the present embodiments is also specified.

A computer program according to the present embodiments and a computer-readable storage medium according to the present embodiments may also be respective computer program products with the commands.

Further features of the invention may be derived from the claims, the figures, and the description of the figures. The features and feature combinations mentioned above in the description and the features and feature combinations mentioned below in the description of the figures and/or shown in the figures may be encompassed by the invention not only in the specified combination in each case, but also in other combinations. For example, the invention also encompasses embodiments and feature combinations that do not have all the features of a claim as originally formulated. Further, the invention encompasses embodiments and feature combinations that extend beyond or deviate from the feature combinations described in the back references in the claims.

The invention is explained in more detail below with reference to specific exemplary embodiments and associated schematic drawings. In the figures, same or functionally same elements may be provided with the same reference numbers. The description of same or functionally same elements may not necessarily be repeated with respect to different figures.

### BRIEF DESCRIPTION OF THE DRAWINGS

FIG. 1 shows a schematic representation of an embodiment of an imaging device;

FIG. 2 shows a schematic representation of an object that is mapped by an embodiment of an imaging method; and

FIG. 3 shows a schematic representation of a further object that is mapped by a further exemplary embodiment of an imaging method.

### DETAILED DESCRIPTION

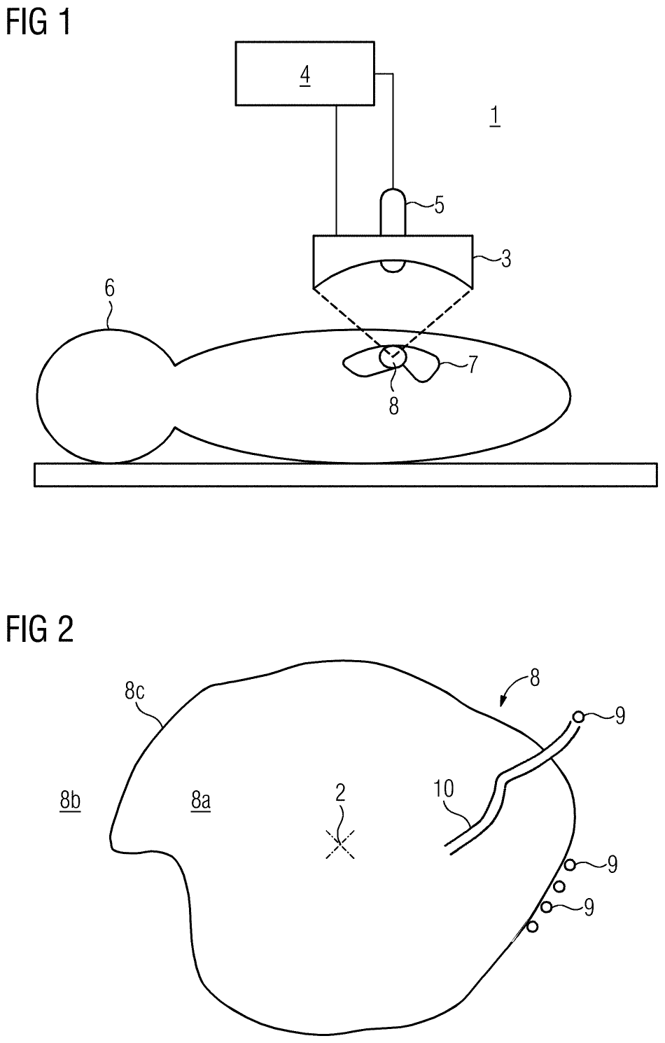

FIG. 1 shows a schematic of an embodiment of an imaging device 1.

The imaging device 1 has an imaging modality 5 (e.g., a sonography system for ultrasound-based imaging). In alternative embodiments, the imaging modality 5 may, however, be configured as a computed tomography device or magnetic resonance tomography device, for example.

The imaging device 1 also has at least one ultrasound source 3 that may be part of a histotripsy device, for example.

The imaging device 1 has one or more controllers, computing units, and/or devices for data processing, and is referred to and shown below as a shared controller 4, in order to simplify the representation and the description.

The controller is therefore configured to control both the at least one ultrasound source 3 and also the imaging modality 5, or obtain images or signals generated by the imaging modality 5. In various embodiments, the different objects and functions of the controller 4 may, however, as mentioned above, be divided over a number of control units, computing units, and/or devices for data processing.

The controller 4 may adjust at least one configuration parameter in order to control the at least one ultrasound source 3, so that the at least one ultrasound source 3 may irradiate ultrasound pulses according to the configuration parameter into a tissue area 7 of a patient 6. The ultrasound pulses merge, for example, in a focus area, the center of which is located in the inner area of an object 8 inside the tissue area 7. The object 8 may be, for example, a tumor or a vessel or a nidus or other vascular malformation.

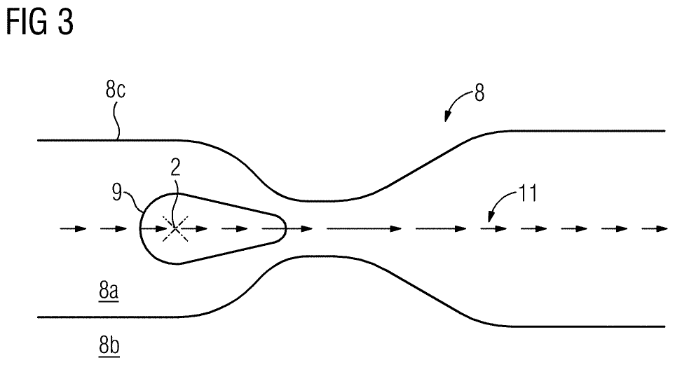

Further, the imaging device 1 contains a positioning system (not shown) that may likewise be controlled by the controller 4 and is configured, controlled by the controller 4, to position the center 2 of the focus area (e.g., in the inner area 8a of the object 8), as shown schematically in FIGS. 2 and 3.

The object 8 or the inner area 8a of the object 8 is separated by a tissue boundary 8c from an outer area 8b, within the tissue area 7, at least partially surrounding the object 8.

The controller 4 is configured to adjust the at least one configuration parameter such that stable cavitation bubbles 9 are generated, as shown in FIG. 2 and FIG. 3, by the irradiated ultrasound pulses in a liquid in the tissue area 7 (e.g., in the inner area 8a and/or the outer area 8b).

The irradiated ultrasound pulses cause a pressure field (e.g., an inhomogenous pressure field) in the tissue area 7, which has a minimum in the center 2 of the focus area. This pressure field exerts a force on the cavitation bubbles 9, and this force causes a specific spatial distribution and/or movement of the plurality of cavitation bubbles 9 to develop, which is caused by the force and the anatomical conditions in the tissue area 7 (e.g., the anatomical structure and shape of the object 8 or the tissue boundary 8c), and possibly also by further forces acting on the cavitation bubbles 9 (e.g., by liquids flowing in the tissue area 7).

The controller 4 may control the imaging modality 5 accordingly such that the imaging modality 5 maps the tissue area 7 at least partially and as a result maps the developing spatial distribution and/or movement of the plurality of cavitation bubbles 9.

In the example in FIG. 2, the object 8 is a tumor, for example. The cavitation bubbles 9 are generated, for example, so that the cavitation bubbles 9 develop exclusively or preferably in the outer area 8b. On account of the low pressure that is strongest in the center 2 of the focus area, the cavitation bubbles 9 outside of the tissue boundary 8c are also drawn in the direction of the center 2 of the focus area and adsorb accordingly to the exterior of the tissue boundary 8c in the outer area 8b, since the cavitation bubbles 9 cannot pass through the tissue boundary 8c. The shape and structure of the tissue boundary 8c may therefore be reproduced by mapping the cavitation bubbles 9 using the imaging modality 5.

For example, tumors or also vascular malformations may have a network-type vascular structure, where it may be important for accurate modeling and/or treatment planning to identify or differentiate supplying and discharging vessels. Such a vessel 10, which passes through the tissue boundary 8c, is shown schematically in FIG. 2.

Cavitation bubbles 9 that are located in the vessel 10 are therefore subjected not solely to the force caused by the low pressure in the center 2 of the focus area, but also to a force caused by the inflowing or outflowing liquid. If the imaging is now carried out by the controller using the imaging modality 5 in a time-resolved manner, then the movement of such cavitation bubbles 9 in the direction of the inner area 8a or away from the inner area 8c may be observed, and as a result, it is possible to infer the flow direction of the liquid in the vessel 10.

From the result of the imaging, it may be possible to determine not only the direction of the flow in the vessel 10 in qualitative terms but also the flow speed in the vessel 10 in quantitative or semiquantitative terms.

A blood vessel is shown schematically as the object 8 in FIG. 3. The cavitation bubbles 9 are generated by a corresponding adaptation or adjustment of the configuration parameters at least partially in the inner area 8a (e.g., exclusively in the inner area 8a). In the inner area 8a of the object 8, a liquid 11 (e.g., blood) flows from left to right in the example shown in FIG. 3. As explained above, the cavitation bubbles 9 that develop about the center 2 of the focus area are subjected to a force in the direction of the center 2 on account of the low pressure. By the liquid flow of the liquid 11 in the vessel (e.g., the blood flow), the cavitation bubbles 9 are moved downstream of the center 2 away from the center 2 or upstream toward the center 2 of the focus area. The cavitation bubbles 9 may, for example, dissipate again downstream on account of the pressure increasing with the distance from the center 2.

Based on the at least one configuration parameter, the strength of the low pressure in the center 2, the rate of generation of the cavitation bubbles 9 and the size of the cavitation bubbles 9 may be adapted. For example, the configuration parameters may be adapted so that the cavitation bubbles 9 remain spatially stationary downstream of the center 2. In this case, the forces are to some extent balanced out on account of the low pressure in the center 2 and the flow of the liquid 11. Using the at least one configuration parameter, it is then possible to infer the flow parameters (e.g., the flow speed and/or flow direction) of the liquid 11 and/or other fluid properties of the liquid 11 (e.g., a viscosity).

In various embodiments, differences in the flow parameters or fluid parameters may be identified and determined in a spatially resolved manner by a movement of the center 2 along the vessel. For example, the center 2 may be moved along the vessel. If a spatially stationary distribution of the cavitation bubbles 9 was adjusted as described, it is then possible to infer, for example, with a resolution or change in the stationary distribution that with constant configuration parameters, the force on the cavitation bubbles 9 has changed on account of the flow of liquid 11. It is therefore possible to infer the corresponding changes in the flow parameters or fluid parameters, such as may occur, for example, in vascular restrictions or stenoses.

As described, for example, on the basis of the figures, the present embodiments allow for contrast agent to be dispensed with in an imaging method and at the same time for tissue boundaries and/or flow parameters or fluid properties to be identified or quantified.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An imaging method for mapping a tissue area of a human or an animal, the imaging method comprising:
generating, in a liquid, in the tissue area, a plurality of cavitation bubbles, the generating of the plurality of cavitation bubbles comprising irradiating ultrasound pulses into the tissue area using at least one ultrasound source;
positioning a center of a focus area of the irradiated ultrasound pulses within a first subarea of the tissue area, wherein the first subarea is separated from a second subarea of the tissue area by a tissue boundary, a flow of liquid is present in the first subarea, or a combination thereof; and
mapping a spatial distribution, movement of the plurality of cavitation bubbles developing in the tissue area on account of a pressure field caused by the irradiated ultrasound pulses, or the spatial distribution and the movement of the plurality of cavitation bubbles using an imaging modality.

2. The imaging method of claim 1, wherein the mapping of the spatial distribution, the movement of the plurality of cavitation bubbles, or the spatial distribution and the movement of the plurality of cavitation bubbles includes an x-ray based imaging, a magnetic resonance tomography, or an ultrasound-based imaging.

3. The imaging method of claim 2, wherein the mapping of the spatial distribution, the movement of the plurality of cavitation bubbles, or the spatial distribution and the movement of the plurality of cavitation bubbles is carried out without administration of contrast agent.

4. The imaging method of claim 1, wherein the first subarea is separated from the second subarea by the tissue boundary, and
wherein the first subarea corresponds to an inner area of an object within the tissue area, and the second subarea corresponds to an outer area surrounding the object at least partially.

5. The imaging method of claim 4, wherein the ultrasound pulses are generated such that the pressure field has a minimum in the inner area.

6. The imaging method of claim 4, wherein the ultrasound pulses are generated such that at least one part of the plurality of cavitation bubbles is generated in the outer area.

7. The imaging method of claim 6, further comprising determining, based on a result of the mapping of the movement of the plurality of cavitation bubbles, a movement direction, a movement speed, or the movement direction and the movement speed of one or more cavitation bubbles of the at least one part of the plurality of cavitation bubbles generated in the outer area through a vessel, and
wherein the vessel extends through the tissue boundary and fluidically connects the inner area with the outer area.

8. The imaging method of claim 4, wherein the ultrasound pulses are generated such that at least one part of the plurality of cavitation bubbles is generated in the outer area.

9. The imaging method of claim 4, wherein the first subarea is separated from the second subarea by the tissue boundary, and
wherein the flow of liquid is present in the first subarea, and the ultrasound pulses are generated such that at least one part of the plurality of cavitation bubbles is generated in the inner area.

10. The imaging method of claim 9, further comprising determining a flow direction, a flow speed, a viscosity, or any combination thereof of the liquid in the inner area based on a result of the mapping of the spatial arrangement, the movement of the plurality of cavitation bubbles, or the spatial arrangement and the movement of the plurality of cavitation bubbles.

11. The imaging method of claim 10, wherein the position of the center of the focus area within the inner area is moved, and
wherein the flow direction, the flow speed, the viscosity, or the respective combination thereof within the inner area is determined in a location-dependent manner.

12. An imaging device for mapping a tissue area of a human or an animal, the imaging device comprising:
an imaging modality comprising:
at least one ultrasound source; and
a positioning system for the at least one ultrasound source; and
a controller configured to:
control the at least one ultrasound source, such that ultrasound pulses are irradiated into the tissue area; and
adjust at least one configuration parameter for controlling the at least one ultrasound source, such that a plurality of cavitation bubbles is generated by the irradiated ultrasound pulses in a liquid in the tissue area,
wherein the positioning system is configured to position a center of a focus area of the irradiated ultrasound pulses within a first subarea of the tissue area, wherein the first subarea is separated from a second subarea of the tissue area by a tissue boundary, a flow of liquid is present in the first subarea, or a combination thereof, and
wherein the imaging modality is configured to map a spatial distribution, movement of the plurality of cavitation bubbles developing in the tissue area on account of a pressure field caused by the irradiated ultrasound pulses, or the spatial distribution and the movement of the plurality of cavitation bubbles.

13. The imaging device of claim 12, further comprising at least one histotripsy converter that includes the at least one ultrasound source.

14. The imaging device of claim 12, wherein the imaging modality is configured as an x-ray-based imaging modality, as a magnetic resonance tomography device, or as an ultrasound-based imaging modality.

15. In a non-transitory computer-readable storage medium that stores instructions executable by an imaging device to map a tissue area of a human or an animal, the instructions comprising:
generating, in a liquid, in the tissue area, a plurality of cavitation bubbles, the generating of the plurality of cavitation bubbles comprising irradiating ultrasound pulses into the tissue area using at least one ultrasound source;
positioning a center of a focus area of the irradiated ultrasound pulses within a first subarea of the tissue area, wherein the first subarea is separated from a second subarea of the tissue area by a tissue boundary, a flow of liquid is present in the first subarea, or a combination thereof; and
mapping a spatial distribution, movement of the plurality of cavitation bubbles developing in the tissue area, or the spatial distribution and the movement of the plurality of cavitation bubbles on account of a pressure field caused by the irradiated ultrasound pulses using an imaging modality.

16. The non-transitory computer-readable storage medium of claim 15, wherein the mapping of the spatial distribution, the movement of the plurality of cavitation bubbles, or the spatial distribution and the movement of the plurality of cavitation bubbles includes an x-ray based imaging, a magnetic resonance tomography, or an ultrasound-based imaging.

17. The non-transitory computer-readable storage medium of claim 16, wherein the mapping of the spatial distribution, the movement of the plurality of cavitation bubbles, or the spatial distribution and the movement of the plurality of cavitation bubbles is carried out without administration of contrast agent.

18. The non-transitory computer-readable storage medium of claim 15, wherein the first subarea is separated from the second subarea by the tissue boundary, and
wherein the first subarea corresponds to an inner area of an object within the tissue area, and the second subarea corresponds to an outer area surrounding the object at least partially.
